(19) United States
(12) Patent Application Publication
(10) Pub. No.: US 2007/0141562 A1
(43) Pub. Date: Jun. 21, 2007

(54) Apparatus and methods for high yield microbial surface sampling

(76) Inventor: Thomas H. Grove, Manhattan Beach, CA (US)

Correspondence Address: Thomas H. Grove, 632 26th Street, Manhattan Beach, CA, 90266, US

(21) Appl. No.: 11/636,093

(22) Filed: Dec. 8, 2006

Related U.S. Application Data
(60) Provisional application No. 60/750,119, filed on Dec. 15, 2005.

(51) Int. Cl. C12Q 1/70; C12Q 1/04; B32B 27/04; C12M 1/34; C12M 3/00

(52) U.S. Cl. 435/5; 435/34; 435/287.1; 442/123

(57) ABSTRACT

This invention discloses an apparatus and methods for high yield microbial surface sampling. The microbial sampling techniques in which a sample is obtained from an environmental or biological surface using a microfiber material in the form of microfiber devices, wipes, or swabs.

### CROSS-REFERENCE TO RELATED APPLICATION

This application claims priority of a U.S. provisional application Ser. No. 60/750,119, filed Dec. 15, 2005, entitled “Apparatus And Methods For High Yield Microbial Surface Sampling”, the entire contents of the co-pending application are incorporated herein by reference.

### FIELD OF THE INVENTION

The present invention relates generally to microbial sampling techniques and, more specifically, to materials and processes that collect and recover a significantly greater amount of microbes, such as bacteria, viruses and fungi, during sample collection compared to traditional sampling devices. Particularly, the present invention relates to microbial sampling techniques in which a sample is obtained from an environmental or biological surface using a microfiber material in the form of microfiber devices, wipes, or swabs. The microbes captured on the microfiber can be eluted from the microfiber material for further repair, growth and detection.

### BACKGROUND OF THE INVENTION

When surfaces become contaminated with bacteria, fungi, molds, yeasts, viruses, or other microorganisms (“microbes”), sickness (morbidity) and, sometimes, death (mortality) may result. This is particularly true when body surfaces or cavities, such as skin or an infected throat, or environmental surfaces, such as in food processing plants and healthcare facilitates become contaminated with microorganisms.

In medical practice, body surfaces and/or cavities may be infected with bacteria, fungi, viruses or other microbes and cause aliments such as respiratory, venereal or skin diseases. For example, beta-hemolytic group A Streptococcus is a major cause of upper respiratory infection such as tonsillitis, pharyngitis and scarlet fever. Early diagnosis and treatment of group A streptococcal pharyngitis has been shown to reduce the severity of symptoms and further complications, such as rheumatic fever and glomerulonephritis. Recent development of immunological techniques that can detect group A streptococcal antigen directly from throat swabs, allows physicians to diagnose and administer therapy immediately. To perform the test, a throat swab specimen is collected using a sterile Dacron or Rayon tipped swab. Target antigen is extracted from the swab specimen and detected using a lateral flow device or equivalent detection platform. The sensitivity of the test relies greatly on how efficiently the swab collects the bacteria from the throat surface and how effectively the target antigen is released from the collection device prior to detection.

Environmental microbial contaminations is also a concern in healthcare facilities since some of the patients of such facilities often suffer from infections by pathogenic microbes and, thus, bring the pathogenic microbes in proximity to other patients. Once an environmental surface has become contaminated with microbes, contact with the contaminated surface may easily and readily transfer microbes to other locations, such as another surface, an individual, equipment, or the like and lead to nosicomal infections at the healthcare facility.

In industry, food processing plant surfaces (e.g., solid surfaces, equipment surfaces, protective clothing, etc.) may become contaminated during food production. Such contamination may be caused by or transferred to meat or other foods. As is well known, microbial contamination and transfer in certain environments may pose significant health risks. For example, the food that leaves a contaminated food processing plant will subsequently be consumed, and may cause sickness and, possibly, death. Microorganisms such as Listeria monocytogenes and Escherichia coli O157:H7 are of particular concern. L. monocytogenes grows even when refrigerated, while E. coli O157:H7 infections are aggressive and often deadly.

In view of the potential dangers of microbial contamination, in particular the ease with which microbes may be transferred in certain environments and the health hazards associated with the contamination of certain environments, a variety of techniques have been developed and employed to detect such contamination so that it may be promptly decontaminated. Key to all of these techniques is the efficient collection and recovery of microbes from a surface or cavity being investigated.

### SUMMARY OF THE INVENTION

Some aspects of the invention provide a method for high yield microbial surface sampling comprising: providing a sterile sampling device made of microfibers, contacting the device on a surface to collect microbes, culturing the collected microbes to provide a sufficient number of organisms that are suitable for further analysis. It is suggested that the sampling device or apparatus made of microfibers has more surface areas to collect the microbes than other sampling device made of conventional fibers. In one embodiment, the microfibers weigh 1 denier or less. In another embodiment, the outside diameter of the microfibers is about 1,000 nanometers or less, preferably 500 nanometers or less.

Some aspects of the invention provide a system for high yield microbial surface sampling comprises a sterile sampling device made of microfibers, sterile Letheen broth, and a Whirl-Pak™ bag. In one embodiment, the sterile sampling device is pyrogen-free.

In some embodiments, the microfibers are made of material selected from a group consisting of acrylic, nylon, polyester, rayon, and combinations thereof. In a further embodiment, the sampling device comprises a core and an outer skin, wherein the device is created by combining polyamide microfibers as the core and polyester microfibers as the outer skin of the device. In still another embodiment, the polyamide microfibers constitute about 10 to 40% of the device, preferably about 10-20% of the device.

Some aspects of the invention provide a method for high yield microbial surface sampling, wherein the sampling includes collection of bacteria, fungi, molds, yeasts, viruses, or microorganisms from the surface, and wherein the surface may be an internal surface of a cavity.

Some aspects of the invention provide a method for high yield microbial surface sampling comprising providing a sterile sampling device made of microfibers, contacting the device on a surface to collect microbes, culturing the collected microbes to provide a sufficient number of organisms that are suitable for further analysis, wherein the culturing step is loaded with a growth medium to stimulate growth of the microbes in an incubator. Further, the growth medium includes ingredients that will allow some microorganisms to grow at much faster rates than other microbes. In one embodiment, the culturing step in the incubator is for a period of about 1 to about 72 hours, preferably about 6 to about 48 hours.

In one embodiment, the sampling device is in the form of wipes, swabs, Q-tip, or a wipe tip with an ergonomic handle.

For purposes of summarizing the invention, certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as conceived or suggested herein without necessarily achieving other advantages as may be conceived or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 shows nonwoven microfiber, towelette wipes, one-ply composite tissue and sponge.

FIG. 2 shows recovery of Listeria monocytogenes from stainless steel surfaces by four different sample collection devices: MF is microfiber wipe; TMI is towelette; KW is Kimwipes; and SP is cellulose sponge. (A) initial inoculum 5.04 CFU/10 cm²; (B) initial inoculum 4.04 CFU/10 cm²; (C) initial inoculum 3.04 CFU/10 cm²; and (D) initial inoculum 2.04 CFU/10 cm¹.

### DETAILED DESCRIPTION OF EXEMPLARY EMBODIMENTS

The apparatus and process of the present sample collection invention may be used for a variety of applications, including, but not limited to, food testing, water testing, medical and veterinary testing and evaluation, and forensic testing.

By way of nonlimiting example, teachings of the present invention may be used to collect bacteria, fungi, molds, yeasts, viruses, or other microorganisms from a surface or cavity. The apparatus and processes of the present invention may, for example, be used to collect bacteria of one or more of the genuses Listeria, Campylobacter, Escherichia, Salmonella, Clostridia, Shigella, Staphylococcus, Vibrio, Yersinia, Plesiomonas, Bacillus, Streptococcus, Neisseria, Enterococcus, Enterobacter, Citrobacter, Vibrio, Legionella, Haemophilus, Pseudomonas, Gardnerella, Francisella, Brucella, Bordetella, Borrelia, Mycobacterium, Nocardia, and Aeromonas from a surface, cavity, or concealed space. Samples may be collected for yeasts, including, without limitation, yeasts of one or more of the genuses Kluyveromyces, Pichia, Saccharomyces, Candida, and Rhodotorula. Alternatively, or in addition, samples may be collected for molds, including, but not limited to, molds of the genuses Byssochlamys, Fusarium, Geotrichium, Penicillum, Aspergillosis, and Scopulariopsis.

Microfiber is the terminology used to describe ultra-fine manufactured fibers and the name given to the technology of developing these fibers. Fibers made using microfiber technology, produce fibers, which weigh less than 1 denier. Denier is defined as the mass in grams per 9000 meters. The higher the number, the thicker or denser the fiber. Many microfibers are 0.5 to 0.6 denier. In one embodiment, the sampling apparatus with microfibers at less than 0.6 denier is particularly feasible for the current application. For another comparison, very fine nylon stockings are knit from 10 to 15 denier yarns consisting of 3 to 4 filaments. A 15 denier yarn made of microfibers would have as many as 30 filaments. The fabrics made from these extra-fine fibers provide superior absorbent properties. Currently, there are at least four types of synthetic microfibers being produced. These include acrylic, nylon, polyester and rayon or combinations of these fibers. In an alternate embodiment, the diameter of the microfibers is about 1,000 nanometers or less, preferably 500 nanometers or less, and most preferably 250 nanometers or less. In another embodiment, the microfibers in the apparatus for high yield microbial sampling are about the same deniers or diameters. In still another embodiment, the sampling apparatus comprises microfibers with a wide spectrum of weight (about 0.01 denier to about 1 denier) and diameters (about 10 nanometers to about 1000 nanometers) to pick up more microbes (in quantity) or more varieties of microbes.

In one embodiment, microfiber is created by combining two DuPont fiber inventions: polyester and polyamide (nylon). The polyamide is used as the core of the hybrid fiber (generally 10 to 40% of the content) and the polyester is the outer skin (60 to 90%). Each fiber has specific qualities that, when properly blended, can be used to weave functionally specific fabrics. In one alternate embodiment, the polyamide component constitutes 5-20% (preferably 10-20%) of the composite microfiber with the balance of polyester component. In one embodiment, the average diameter of a microfiber is about 100 to 500 nanometers.

Microfiber yarn was developed in the 1980s to stimulate competition for natural yarn materials, like cotton and silk. One of the early adopters of microfiber yarn was Olsson Cleaning Technology, Sweden, who discovered that splitting the fibers made the fibers “grab” and improved the performance of cleaning towels. By 1994, the semiconductor industry was introduced to microfiber cleaning cloths, which could be used to wipe down the clean rooms used to produce memory, computer processors and other microchips. The present invention takes advantage of the binding properties of microfiber to collect, transport and process microbes for further enrichment and detection.

Environmental Sampling

Conventionally, environmental microbial testing initially includes obtaining a sample from a surface. This is typically done by contacting (e.g., wiping, swiping, etc.) the surface with a sterile sampling appliance, such as a Dacron, Rayon, Calcium alginate or cotton swab, a cellulose sponge or a composite tissue. Environmental surfaces that are tested in this manner are usually quite clean; thus, the number of microorganisms that are picked up by the sampling appliance is typically quite low. Due to the small sample size, microbes such as bacteria, yeast and fungi that are on (e.g., picked up by) the sampling appliance must be reproduced, or “grown” or “cultured,” to provide a sufficient number of organisms that are suitable for further analysis. Accordingly, the sample is then typically neutralized and, optionally, stabilized, repaired, or enriched, then applied (e.g., swiping, dipping, and agitating, etc.) to an appropriate growth medium (e.g., agar (a gelatin or gelatin-like material), broth (a liquid), etc.), which includes nutrients that will help microbes of interest to grow. The growth medium, used to stimulate growth of the microbes, may be selective, meaning that the growth medium may include ingredients that will allow some microorganisms to grow at much faster rates than other microbes or ingredients that will prevent the growth of at least some undesired microbes. The growth medium is incubated or held at a certain temperature for a predetermined period of time—typically about 6 to about 48 hours—or until microbial growth is visibly apparent. In one embodiment with quick sampling requirements, the incubation period may be as short as 1 hour in appropriate growth media and temperature.

The present invention uses microfiber wipes, swabs, or a wipe tip with an ergonomic handle to replace traditional sampling devices. In the example described below, sterile microfiber wipes (3 cm by 6 cm) were rehydrated with 200 μL Letheen broth and used to collect Listeria bacteria from stainless steel environmental surfaces.

### EXAMPLE 1

### Preparation of Samples for Environmental Listeria Testing

Cultures and cell suspension. The test cultures, Listeria monocytogenes ATCC 51776 and Listeria welshimeri ATCC 35897, were obtained from American Type Culture Collection (Manassas, Va.). Cultures and maintained at −25° C. in Trypticase soy broth (Difco, Becton Dickinson, Sparks, Md.) containing 10 % (vol/vol) glycerol until revived. Trypticase soy agar containing 0.6% yeast extract (TSAYE) (Difco) was inoculated from frozen stock culture and incubated at 37° C. for 24 hours. Single colonies on TSAYE from each culture were transferred into 10 mL Trypticase soy broth with 0.6% yeast extract (TSBYE), and incubated at 37° C. for 24 hours. Cultures were centrifuged at 10,000 X g for 10 minutes at room temperature. Pellets were resuspended in buffered peptone water (BPW). Cell concentration was determined by adjusting optical densities of each culture at 600 nm to 1.00 (±0.05), and preparing serial 10-fold dilutions with BPW and plating on TSAYE after 24 hours of growth at 37° C. All bacteriological media used in this study were from Difco (Detroit, Mich.) unless stated otherwise.

Stainless steel surfaces. A stainless steel plate was marked and divided into 48 equal 10 cm by 10 cm squares. Then, the plate was disinfected by using 10% chlorine, and washed three-times with sterile distilled water. The non-chlorine treated and chlorine-treated surfaces were both used in the experiments to determine the effect of any possible residual chlorine on the surface against test microorganisms.

Environmental Listeria recovery devices. Four recovery devices were used throughout the study—sterile cellulose sponges (SP; Nasco Speci-Sponges, Nasco, Fort Atkinson, Wis.; 4 cm by 8 cm)), sterile microfiber wipes (MF; Furuisen, China; 70% polyester and 30% polyamide, 130 g/m²; 3 cm by 6 cm), sterile towelette wipe (TMI; TMI Upland, Calif.; 70% polypropylene and 30% rayon), and a sterile one-ply composite tissue (CT; Kim-Wipes® EX-L 1-ply wipers, 11.4 cm by 21.3 cm; Kimberly-Clark Corp., Roswell, Ga.).

Inoculation of Listeria species on environmental surfaces. Adjusted cell suspensions were 10-fold serially diluted with BPW and 100 μL cell suspensions were spotted on stainless steel surfaces, uniformly distributed over with a sterile pipette tip to obtain inoculum levels of ˜10⁵, 10⁴, 10³, and 10² CFU/square. Each square was marked as 10 cm by 10 cm. In this study, inoculated plates were allowed to dry at room temperature (−23° C.) for 24 hours.

Rehydration of environmental recovery devices. All environmental recovery devices were rehydrated with sterile Letheen broth. MF and TMI were rehydrated with 200 μL Letheen broth in 2-oz Whirl-Pak bag. SP was rehydrated with 5 mL Letheen broth in 18-oz Whirl-Pak bag and CT was rehydrated with 0.5 mL Letheen broth in 2-oz Whirl-Pak bag, respectively. Then, using disposable gloves, stainless steel surfaces were swabbed 15 times horizontally and 15 times vertically with the environmental recovery devices.

Repair period. After wiping, each MF, TMI, CT and SP sample was returned to the same Whirl-Pak bag previously used to store and hydrate each sampling device. The sampling devices were then rehydrated in order to assist in the recovery of microbes from the sample device. For MF and TMI, 1.8 mL of Letheen broth was added into Whirl-Pak bags for rehydration (total volume of Letheen broth was 2 mL). Additional 5 mL and 1.5 mL Letheen broth were added into Whirl-Pak bags for SP and CT, respectively. The total volume of Letheen broth added into Whirl-Pak bags for SP was 10 mL, and it was 2 mL for CT. After adding Letheen broth, swabbed samples were kept at room temperature for 2 hours for repair and recovery.

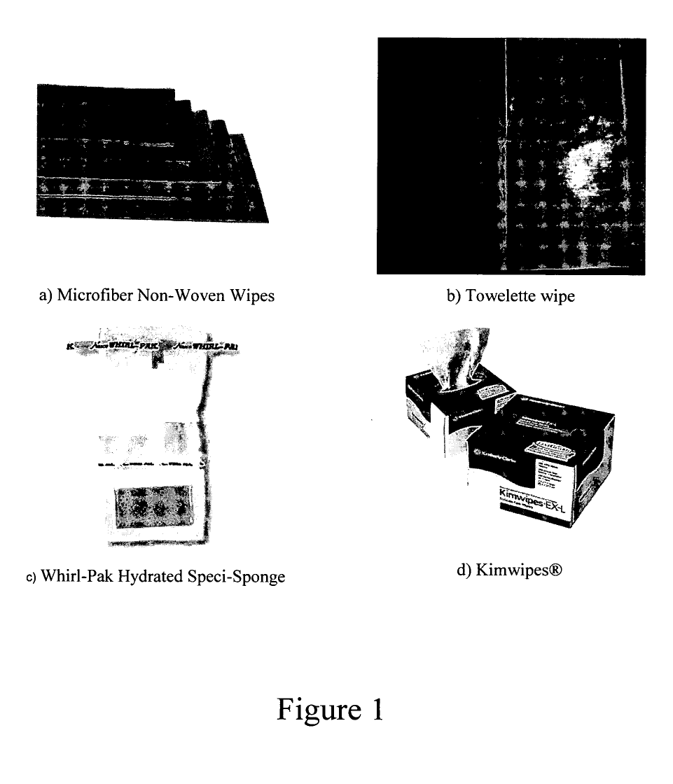

Results. The essential function of the proposed sample collection technology is to use microfiber material in the form of a wipe or swab to collect microbes from environmental surfaces or cavities. Ideally, a sample collection device should be able to remove microbes from environmental surfaces or cavities and transfer the microbes to liquid broth for further enrichment of the microbe population of interest. FIG. 1 shows the proposed microfiber material along with three other materials in current use. The most common material in use is the cellulose sponge. One aspect of the invention provides a sterile, pyrogen-free sampling device made of microfibers.

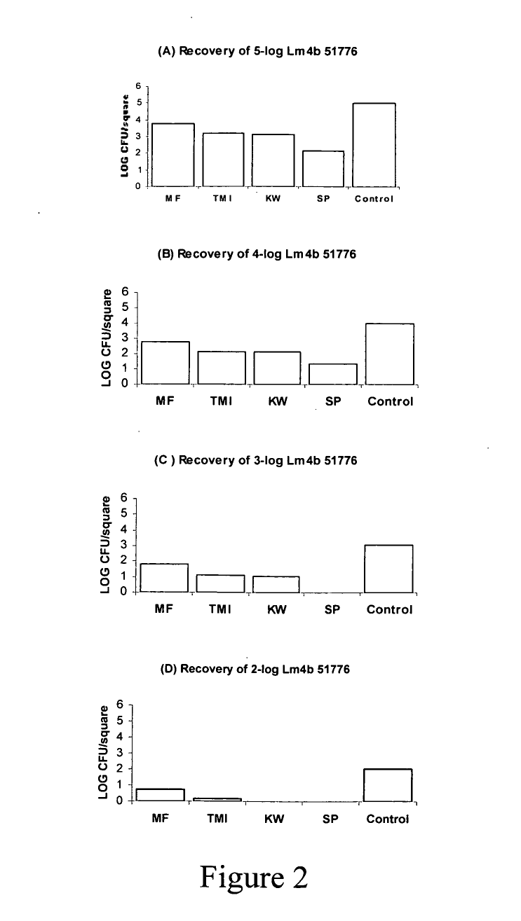

FIG. 2 shows recovery of Listeria monocytogenes from stainless steel surfaces by four different sample collection devices: MF is microfiber wipe; TMI is towelette; KW is Kimwipes; and SP is cellulose sponge. (A) initial inoculum 5.04 CFU/10 cm²; (B) initial inoculum 4.04 CFU/10 cm²; (C) initial inoculum 3.04 CFU/10 cm²; and (D) initial inoculum 2.04 CFU/10 cm². Microfiber wipes (MF) yielded the best recovery of the sample collection devices, with populations 0.61 to 1.62 log/10 cm² higher compared with TMI, KW and SP sample collection devices when stainless steel surfaces are populated at 5.04 CFU/10 cm² (FIG. 2 and Table 1). Least square means by average log count were compared for effect of the sample collection device and subject to the least square significant difference test. Differences between MF and the other devices were statistically significant (P<0.05) (Table 1). SP, which is the recommended sample collection device for environmental surface testing, was the least effective of the sampling devices. At levels below 3.04 CFU/10 cm², the SP sampling device did not recover any Listeria from the stainless steel surface. MF consistently achieved 4.2-fold and 5-fold better recovery than TMI and KW sampling devices across the range of Listeria doses.

TABLE 1
Recovery of L. monocytogenes from stainless steel surfaces by four
different sample collection devices.

| I. RECOVERY | | | | |
|---|---|---|---|---|
| DEVICES | II. LOG CFU/10 CM² | | | |
| Control - CFU added | 2.04^{a} | 3.04^{a} | 4.04^{a} | 5.04^{a} |
| to stainless steel | | | | |
| MF | 0.74^{b} | 1.82^{b} | 2.77^{b} | 3.78^{b} |
| TMI | 0.18^{c} | 1.13^{c} | 2.14^{c} | 3.17^{c} |
| KW | <10^{d} | 1.08^{c} | 2.15^{c} | 3.12^{c} |
| SP | ND | <10^{d} | 1.37^{d} | 2.16^{d} |

Note:
ND—Not detected.
Cell numbers were the average of colony numbers from two independent experiments with duplicates.
Different letters are significantly different from each other at the P < 0.05 level.

From the foregoing description, it will be appreciated that microfiber wipes (MF) yielded the best recovery of the sample collection devices, with populations 0.61 to 1.62 log/10 cm² higher compared with TMI, KW and SP sample collection devices when stainless steel surfaces are populated at 5.04 CFU/10 cm² (FIG. 2 and Table 1). While the apparatus, process and aspects of the invention have been described with a certain degree of particularity, it is manifest that many changes may be made in the specific designs, constructions and methodology herein above described without departing from the spirit and scope of this disclosure.

Various modifications and applications of the invention may occur to those who are skilled in the art, without departing from the true spirit or scope of the invention. It should be understood that the invention is not limited to the embodiments set forth herein for purposes of exemplification, but is to be defined only by a fair reading of the appended claims, including the full range of equivalency to which each element thereof is entitled.

What is claimed is:

1. A method for high yield microbial surface sampling comprising: providing a sterile sampling device made of microfibers, contacting said device on a surface to collect microbes, culturing said collected microbes to provide a sufficient number of organisms that are suitable for further analysis.

2. The method of claim 1, wherein the microfibers weigh 1 denier or less.

3. The method of claim 1, wherein diameter of the microfibers is about 1000 nanometers or less.

4. The method of claim 1, wherein the microfibers are made of material selected from a group consisting of acrylic, nylon, polyester, rayon, and combinations thereof.

5. The method of claim 1, wherein the sampling device comprises a core and an outer skin, wherein the device is created by combining polyamide microfibers as the core and polyester microfibers as the outer skin of said device.

6. The method of claim 5, wherein the polyamide microfibers constitute about 10 to 40% of the device.

7. The method of claim 1, wherein the sampling includes collection of bacteria, fungi, molds, yeasts, viruses, or microorganisms from the surface.

8. The method of claim 1, wherein the surface is an internal surface of a cavity or a concealed space.

9. The method of claim 1, wherein the culturing step is enhanced by loading the device with a growth medium to stimulate growth of the microbes in an incubator.

10. The method of claim 9, wherein the growth medium includes at least one ingredient that allows some microorganisms to grow at much faster rates than other microbes.

11. The method of claim 9, wherein the culturing step in the incubator is for a period of about 6 to about 48 hours.

12. The method of claim 1, wherein the sampling device is in a form of wipes, swabs, or a wipe tip with an ergonomic handle.

13. A system for high yield microbial surface sampling comprises a sterile sampling device made of microfibers, sterile Letheen broth, and a Whirl-Pak™ bag.

14. The system of claim 13, wherein the sampling device is in a form of wipes or swabs.

15. The system of claim 13, wherein the microfibers weigh 1 denier or less.

16. The system of claim 13, wherein the microfibers are made of material selected from a group consisting of acrylic, nylon, polyester, rayon, and combinations thereof.

17. The system of claim 13, wherein the sampling device comprises a core and an outer skin, wherein the device is created by combining polyamide microfibers as the core and polyester microfibers as the outer skin of said device.

18. The system of claim 17, wherein the polyamide microfibers constitute about 10 to 40% of the device.

19. The system of claim 13, wherein the sampling device comprises a polyamide component and a polyester component, the polyamide component constituting 10-20% of the sampling device.

20. The system of claim 13, wherein diameter of the microfibers is about 1000 nanometers or less.
